(19) United States
(12) Patent Application Publication
(10) Pub. No.: US 2016/0066887 A1
(43) Pub. Date: Mar. 10, 2016

(54) IMAGE INDICATOR PROVISION IN ULTRASOUND SYSTEM

(71) Applicant: SAMSUNG MEDISON CO., LTD., Hongcheon-gun (KR)

(72) Inventors: Dong-gyu HYUN, Hongcheon-gun (KR); Norio SHINOZUKA, Hongcheon-gun (KR)

(21) Appl. No.: 14/941,510

(22) Filed: Nov. 13, 2015

Related U.S. Application Data
(63) Continuation-in-part of application No. 12/693,347, filed on Jan. 25, 2010, now Pat. No. 9,211,105.
(60) Provisional application No. 62/142,322, filed on Apr. 2, 2015.

(30) Foreign Application Priority Data
Jan. 28, 2009 (KR) 10-2009-0006570

(51) Int. Cl. A61B 8/00; A61B 8/14; A61B 8/08

(52) U.S. Cl. CPC A61B 8/463; A61B 8/5207; A61B 8/14

(57) ABSTRACT

An ultrasound system includes a probe scanning a target object by transmitting ultrasound signals and receiving ultrasound echo signals, a processor forming an ultrasound image based on the ultrasound echo signals and an ultrasound beam direction marker indicating a transmission direction of the ultrasound signals, and a display displaying the ultrasound image and the ultrasound beam direction marker. The processor controls the display such that the display displays a first indicator indicating a reference point of the ultrasound beam direction marker at a side of the ultrasound beam direction marker, and a second indicator indicating a location corresponding to the first indicator at a side of the ultrasound image.

### CROSS-REFERENCE TO RELATED APPLICATIONS

This application claims the benefit of Korean Patent Application No. 10-2009-0006570, filed on Jan. 28, 2009, in the Korean Intellectual Property Office, U.S. patent application Ser. No. 12/693,347, filed on Jan. 25, 2010, and U.S. Provisional Patent application 62/142,322 filed on Apr. 2, 2015, the disclosures of which are incorporated herein in their entirety by reference.

### BACKGROUND

1. Field

The present disclosure relates to an ultrasound system, and more particularly to an ultrasound system that can provide image indicators corresponding to a target object.

2. Description of the Related Art

An ultrasound system has become an important and popular diagnostic tool since it has a wide range of applications. Specifically, due to its non-invasive and non-destructive nature, the ultrasound system has been extensively used in the medical profession. Modern high-performance ultrasound systems and techniques are commonly used to produce two or three-dimensional diagnostic images of internal features of a target object (e.g., human organs).

Generally, the ultrasound system provides a relatively narrow view angle. This is so that scanning is performed for multiple examination locations of a target object to form ultrasound images corresponding to the respective examination locations. The ultrasound images may be outputted by using an echo printer. The examination is then implemented through the outputted ultrasound images. However, it may be difficult to intuitively recognize which part is scanned or which ultrasound image corresponds to an up, down, left or right ultrasound image of the target object. Thus, the ultrasound images may be outputted together with image indicators (e.g., icons) indicative of corresponding examination parts. The image indicators may be also referred to as body markers. The image indicators may be overlaid on the ultrasound images as texts. For example, if a user selects a text button on a control panel provided by the ultrasound system, then a text input window may be activated on the ultrasound image. The user may manipulate a track ball mounted on the control panel to position a cursor on the text input window for text input. Inputting the text may be performed by using a keyboard, which is also mounted on the control panel. However, inputting the text in such fashion may take a long time and greatly inconvenience the user. Also, since the image indicators are directly selected by the user, the image indicators may be incorrectly set.

### SUMMARY

According to an aspect of an exemplary embodiment, an ultrasound system includes a probe scanning a target object by transmitting ultrasound signals and receiving ultrasound echo signals, a processor forming an ultrasound image based on the ultrasound echo signals and an ultrasound beam direction marker indicating a transmission direction of the ultrasound signals, and a display displaying the ultrasound image and the ultrasound beam direction marker. The processor controls the display to display a first indicator indicating a reference point of the ultrasound beam direction marker at a side of the ultrasound beam direction marker, and a second indicator indicating a location corresponding to the first indicator at a side of the ultrasound image.

The display may further display a target object marker indicating the target object, and a display direction of the ultrasound beam direction marker with respect to the target object marker may correspond to a transmission direction of the ultrasound signals with respect to the target object.

The first indicator may be displayed in the ultrasound beam direction marker at a location where the scanning starts or within a predetermined distance from the location where the scanning starts.

The processor may control the display to display the second indicator at a location corresponding to a scan line where the scanning starts, from among a plurality of scan lines comprised in the ultrasound image.

The first indicator may be displayed in the ultrasound beam direction marker at a location where the scanning ends or within a predetermined distance from the location where the scanning ends.

The processor may control the display to display the second indicator at a location corresponding to a scan line where the scanning ends, from among a plurality of scan lines comprised in the ultrasound image.

The first indicator may indicate a direction of the scanning in the ultrasound beam direction marker by using a first arrow.

The second indicator may indicate a direction of the scanning in the ultrasound image by using a second arrow.

The display may display a symmetrically flipped image of the ultrasound image vertically or horizontally, and the second indicator may indicate a location changed according to a direction in which the ultrasound image is symmetrically flipped.

According to an aspect of another exemplary embodiment, a method of displaying an ultrasound image includes scanning a target object by transmitting ultrasound signals and receiving ultrasound echo signals, forming an ultrasound image based on the ultrasound echo signals and an ultrasound beam direction marker indicating a transmission direction of the ultrasound signals, and displaying the ultrasound image and the ultrasound beam direction marker, displaying a first indicator indicating a reference point of the ultrasound beam direction marker at a side of the ultrasound beam direction marker, and displaying a second indicator indicating a location corresponding to the first indicator at a side of the ultrasound image.

### BRIEF DESCRIPTION OF THE DRAWINGS

These and/or other aspects will become apparent and more readily appreciated from the following description of the exemplary embodiments, taken in conjunction with the accompanying drawings in which:

FIG. 1 is a block diagram showing an illustrative embodiment of an ultrasound system;

FIG. 2 is a block diagram showing an illustrative embodiment of an ultrasound data acquisition unit;

FIG. 3 is a block diagram showing an illustrative embodiment of a processing unit;

FIG. 4 is an exemplary diagram showing a mapping table associating a plurality of target objects and examination locations for each of the target objects with image indicators;

FIG. 5 is a schematic diagram showing an example of displaying image indictors together with an ultrasound image;

FIG. 6 is a schematic diagram showing an example of displaying image indicators together with an ultrasound image, wherein their orientation has been adjusted according to position information of an ultrasound probe;

FIG. 7A is a block diagram showing another illustrative embodiment of an ultrasound system;

FIG. 7B is an exemplary diagram showing an illustrative embodiment of an ultrasound image and an ultrasound beam direction marker;

FIGS. 8A to 8E are exemplary diagrams showing a target organ marker, an ultrasound beam direction marker, and an ultrasound image, according to various transmission directions of ultrasound beams;

FIG. 9 is an exemplary diagram showing an illustrative embodiment of an ultrasound image and an ultrasound beam direction marker when the ultrasound image is symmetrically flipped horizontally or vertically;

FIGS. 10A and 10B are exemplary diagrams showing an example of a transmission direction of an ultrasound signal being changed compared to a direction of a probe;

FIG. 11 is a block diagram showing a configuration of an ultrasound diagnosis apparatus according to an embodiment; and

FIG. 12 is a block diagram showing a configuration of a wireless probe according to an embodiment.

### DETAILED DESCRIPTION

The terms used in this specification are those general terms currently widely used in the art in consideration of functions regarding the inventive concept, but the terms may vary according to the intention of those of ordinary skill in the art, precedents, or new technology in the art. Also, some terms may be arbitrarily selected by the applicant, and in this case, the meaning of the selected terms will be described in detail in the detailed description of the present specification. Thus, the terms used herein have to be defined based on the meaning of the terms together with the description throughout the specification.

Throughout the specification, it will also be understood that when a component “includes” an element, unless there is another opposite description thereto, it should be understood that the component does not exclude another element and may further include another element. In addition, terms such as “ . . . unit,” “ . . . module,” or the like refer to units that perform at least one function or operation, and the units may be implemented as hardware or software or as a combination of hardware and software.

Throughout the specification, an “ultrasound image” refers to an image of a target object, which is obtained using ultrasound waves. Furthermore, a “target object” may be a human, an animal, or a part of a human or animal. For example, the target object may be an organ (e.g., the liver, the heart, the womb, the brain, a breast, or the abdomen), a blood vessel, or a combination thereof. Also, the target object may be a phantom. The phantom means a material having a density, an effective atomic number, and a volume that are approximately the same as those of an organism. For example, the phantom may be a spherical phantom having properties similar to a human body.

Throughout the specification, a “user” may be, but is not limited to, a medical expert, for example, a medical doctor, a nurse, a medical laboratory technologist, or a medical imaging expert, or a technician who repairs medical apparatuses.

Hereinafter, exemplary embodiments will be described in detail with reference to the accompanying drawings.

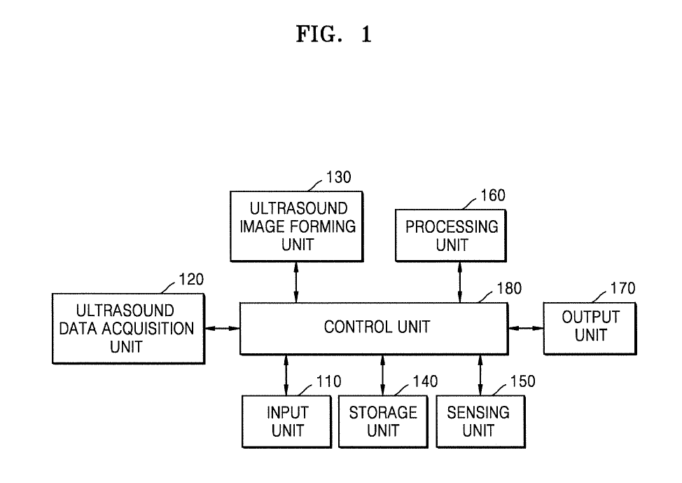

FIG. 1 is a block diagram showing an illustrative embodiment of an ultrasound system 100. As shown therein, the ultrasound system 100 may include an input unit 110 for allowing a user to input instructions. The instructions may include selection instructions for selecting a target object for diagnosis among a plurality of target objects and a specific examination location for the selected target object. The instructions may further include output instructions for requesting an output of the ultrasound image and showing/hiding image indicators on a screen. The target object, examination location and image indicators will be described in detail later. The input unit 110 may include at least one of a control panel, a mouse, a keyboard, a trackball, a touch screen, etc.

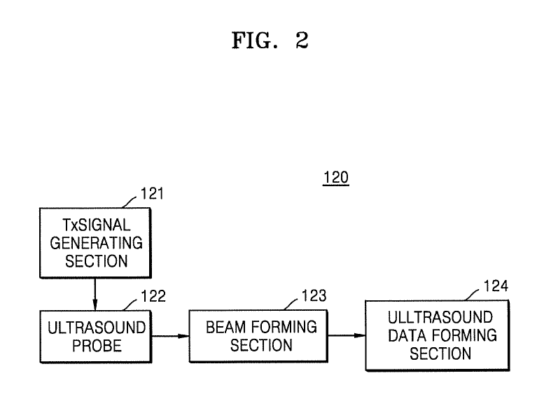

The ultrasound system 100 may further include an ultrasound data acquisition unit 120. The ultrasound data acquisition unit 120 may transmit and receive ultrasound signals to and from the target object to thereby acquire ultrasound data corresponding to a plurality of frames. Referring to FIG. 2, the ultrasound data acquisition unit 120 may include a transmit (Tx) signal generating section 121, which may be configured to generate a plurality of Tx signals.

The ultrasound data acquisition unit 120 may further include an ultrasound probe 122 coupled to the Tx signal generating section 121. The ultrasound probe 122 may transmit the ultrasound signals to the target object in response to the Tx signals. The ultrasound probe 122 may be further configured to receive echo signals reflected from the target object to thereby form electrical receive signals. The ultrasound probe 122 may contain an array transducer consisting of a plurality of transducer elements. In one embodiment, the ultrasound probe 122 may include a convex probe, a linear probe, a 3-dimensional probe, an insertion probe etc., although it is not limited thereto. The insertion probe may include a transvaginal probe and a transrectal probe.

The ultrasound data acquisition unit 120 may further include a beam forming section 123. The beam forming section 123 may apply delays to the electrical receive signals in consideration of positions of the transducer elements and focal points. The beam forming section 123 may further sum the delayed receive signals to thereby output a plurality of receive-focused beams. The ultrasound data acquisition unit 120 may further include an ultrasound data forming section 124, which may form the ultrasound data corresponding to the plurality of frames based on the receive-focused beams. The ultrasound data forming section 124 may be operable to perform signal processing upon the receive-focused beams such as gain adjustment, filtering and the like.

The ultrasound system 100 may further include an ultrasound image forming unit 130 connected to the ultrasound data acquisition unit 120 to receive the ultrasound data. The ultrasound image forming unit 130 may form an ultrasound image of the target object by using the ultrasound data. The ultrasound image may include a brightness-mode image formed by using reflection coefficients of echo signals reflected from the target object, a Doppler-mode image showing spectral Doppler representative of velocities of a moving target object by using the Doppler Effect, a color-mode image showing velocities of moving target objects by using predetermined colors mapped to the respective velocities, an elastic image visualizing mechanical characteristics of tissues based on strain representing deformation of tissues due to the application of the compression and the like.

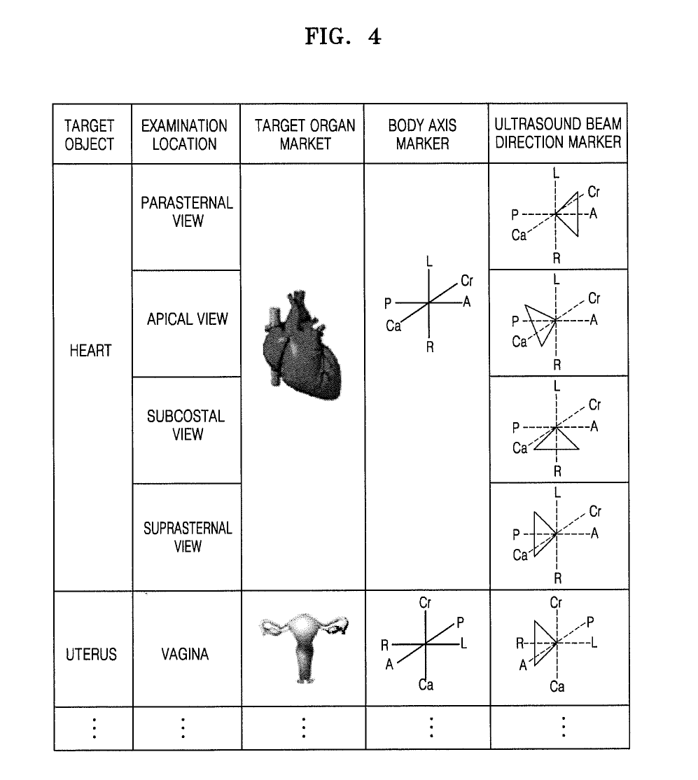

The ultrasound system 100 may further include a storage unit 140, which may store predetermined image indicators corresponding to a plurality of target objects and examination locations for each target object. In one embodiment, the image indicators may include target organ markers indicative of the target objects such as a heart, liver, stomach, uterus, anus and the like. It may also include body axis markers indicative of anatomical orientation of the examination location for each target object such as cranial Cr, caudal Ca, anterior A, posterior P, right R and left L on a 2-dimensional or 3-dimensional coordinate system. The image indicators may further include an ultrasound beam direction marker indicative of a transmission direction of an ultrasound beam transmitted from the ultrasound probe 122 for each examination location. In one embodiment, the storage unit 140 may store a mapping table associating the target objects and examination locations for each target object with the image indicators including the target organ markers, body axis markers and ultrasound beam direction markers, as shown in FIG. 4.

In one embodiment, the target organ markers may be 3-dimensionally or 2-dimensionally represented. Also, the body axis markers may be represented on a 3-dimensional Cartesian coordinate system. Further, the ultrasound beam direction marker may be 2-dimensionally or 3-dimensionally represented according to the type of the ultrasound probe 122. For example, when the ultrasound probe 122 is a 1-dimensional array probe, the ultrasound beam direction marker may be 2-dimensionally represented. Also, when the ultrasound probe 122 is a 2-dimensional array probe or a 3-dimensional mechanical probe, the ultrasound beam direction marker may be 3-dimensionally represented.

The ultrasound system 100 may further include a sensing unit 150, which may sense a position of the ultrasound probe 122 to thereby form 3-dimensional position information of the ultrasound probe 122. The sensing unit 150 may be mounted on a predetermined position of the ultrasound probe 122. Thus, when the ultrasound probe 122 is located in a specific examination location, the sensing unit 150 may sense the 3-dimensional position of the ultrasound probe 122 to form the position information. Any type of sensors capable of sensing a 3-dimensional position of the ultrasound probe 122 may be employed as the sensing unit 150. For example, the sensing unit 150 may include at least one of an angular velocity sensor, magnetic sensor, accelerometer sensor, gravity sensor, Gyro sensor and the like.

The ultrasound system 100 may further include a processing unit 160. The processing unit 160 may access the storage unit 140 to provide the image indicators corresponding to a target object and an examination location selected in response to the instruction inputted by the user. The processing unit 160 may further 3-dimensionally rotate the provided image indicators based on the position information of the ultrasound probe 122, which is formed by the sensing unit 150.

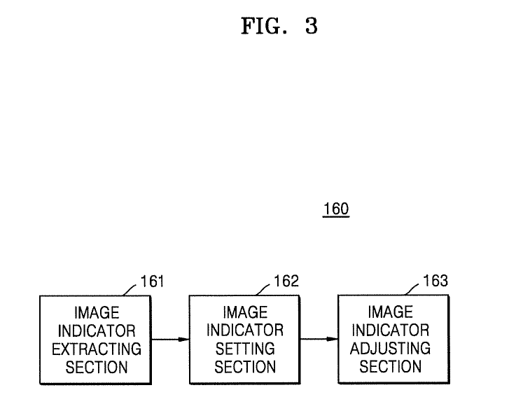

FIG. 3 is a block diagram showing an illustrative embodiment of the processing unit 160. Referring to FIG. 3, the processing unit 160 may include an image indicator extracting section 161. The image indicator extracting section 161 may access the storage unit 140 in response to the selection instruction inputted by the user to extract the image indicators (i.e., target organ marker, body axis marker and ultrasound beam direction marker). For example, if the selection instructions for selecting the uterus as a target object and the vagina as an examination location are inputted through the input unit 110, then the image indicator extracting section 161 may access the storage unit 140 to extract the corresponding image indicators including the target organ marker, body axis marker and ultrasound beam from the mapping table. Also, the selection instructions for selecting the heart as the target object and the parasternal view as the examination location are inputted through the input unit 110, the image indicator extracting section 161 may access the storage unit 140 to extract the image indicators including the target organ marker, body axis marker and ultrasound beam corresponding to the heart and the parasternal view.

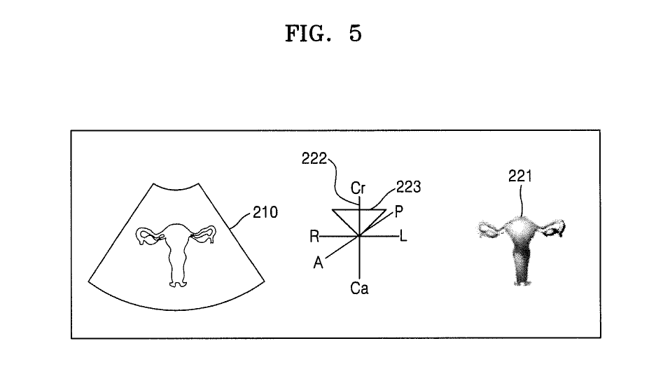

The processing unit 160 may further include an image indicator setting section 162. The image indicator setting section 160 may perform orientation setting of the extracted image indicators based on the position information of the ultrasound probe 122. The image indicators, which are set by the image indicator setting section 160, may be outputted to an output unit 170. The output unit 170 may include a display unit (not shown) such as a CRT monitor, LCD display, OLED display and the like to display the ultrasound image. Further, the output unit 170 may include an echo printer (not shown) to print out the ultrasound image and the image indicators. For example, the image indicator setting section 162 may arrange the extracted body axis marker 222 and ultrasound beam direction marker 223 based on anatomical characteristics of the target object, and set the ultrasound beam direction marker 223 to be overlaid over the body axis marker 222, as shown in FIG. 5. Further, the image indicator setting section 162 may position the target organ marker 221 at the right side of the body axis marker 222 and the ultrasound beam direction marker 223 to perform the orientation setting upon the body axis marker 222, the ultrasound beam direction marker 223 and the target organ marker 221 based on the position information.

Although the above embodiment has been described that the body axis marker is overlaid over the ultrasound beam marker and the target organ marker is positioned at the right side of the body axis marker, the arrangement thereof may not be limited thereto. The body axis marker, the ultrasound beam direction marker and the target organ marker may be set to be overlaid or to be separated from each other.

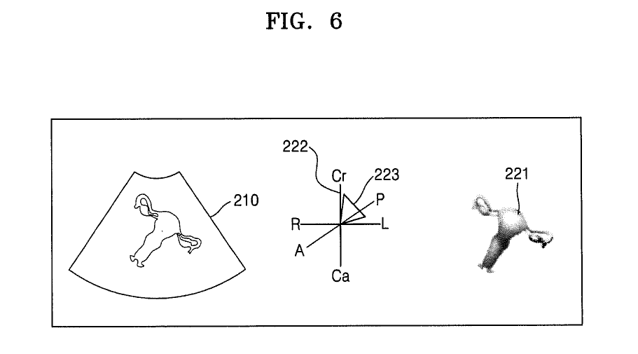

The image processing unit 160 may further include an image indicator adjusting section 163. If the ultrasound probe 122 is moved along a predetermined guide line, then the position information of the ultrasound probe 122 may be changed. The image indicator adjusting section 163 may be configured to adjust the image indicators based on the changed position information. For example, the image indicator adjusting section 163 may rotate the image indicators 3-dimensionally based on the changed position information. The image indicator adjusting section 163 may compute a position difference of the ultrasound probe 122 based on the changed position information, and 3-dimensionally rotate the image indicators including the target organ marker 221, the body axis marker 222 and the ultrasound beam direction marker 223 based on the computed position difference, as illustrated in FIG. 6. In FIGS. 5 and 6, reference numeral “210” may represent an ultrasound image. The image indicator adjusting section 163 may further show or hide the image indicator in response to an instruction for showing/hiding the image indicators on a screen, which may be inputted through the input unit 110.

Referring back to FIG. 1, the ultrasound system 100 may further include a control unit 180. The control unit 180 may control the transmission and reception of the ultrasound signals in the ultrasound data acquisition unit 120 according to an image mode. Further, the control unit 180 may be configured to control entire operations of the elements of the ultrasound system 100.

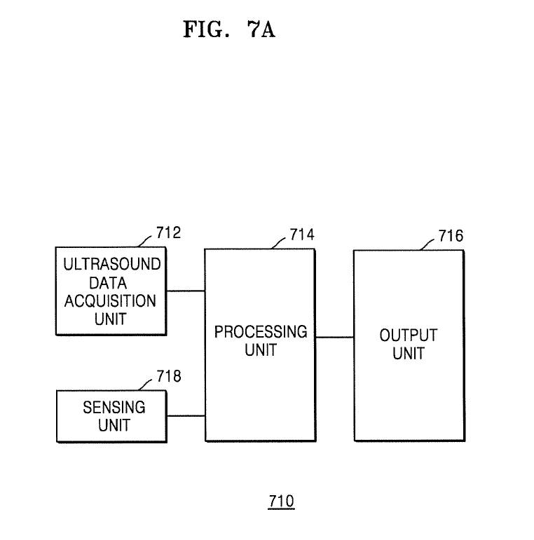

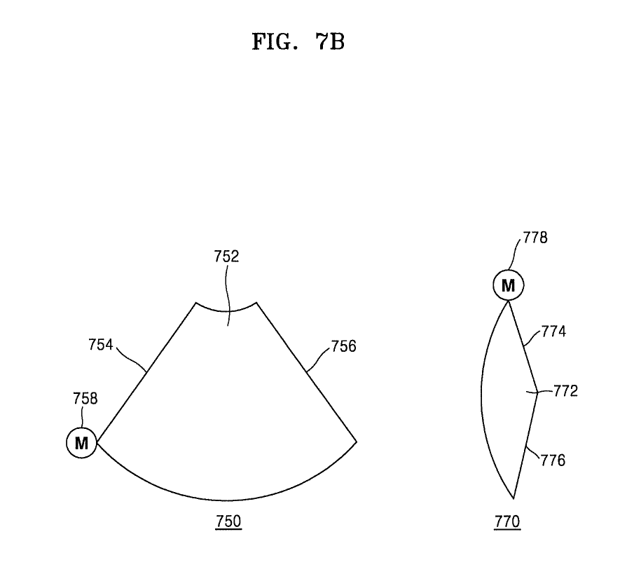

FIG. 7A is a block diagram showing another illustrative embodiment of an ultrasound system 710.

The ultrasound system 710 of FIG. 7A may perform some or all functions performed by the ultrasound system 100 of FIG. 1. In one embodiment, the ultrasound system 710 includes an ultrasound data acquisition unit 712, a processing unit 714, an output unit 716, and a sensing unit 718. The ultrasound data acquisition unit 712, the output unit 716, and the sensing unit 718 of FIG. 7A respectively correspond to the ultrasound data acquisition unit 120, the output unit 170, and the sensing unit 110 of FIG. 1. Also, the processing unit 714 of FIG. 7A may includes some elements or functions of the ultrasound image forming unit 130, the processing unit 160, and the control unit 180 of FIG. 1. The processing unit 714 may include at least one hardware, for example, a central processing unit (CPU), a graphic processing unit (GPU), a microprocessor, or an electronic circuit, but is not limited thereto. Furthermore, the ultrasound data acquisition unit 712 of FIG. 7A may include elements of the Tx signal generating section 121, the ultrasound probe 122 including a plurality of transducer elements, the beam forming section 123, and the ultrasound data forming section 124 shown in FIG. 2.

The sensing unit 718 detects a 3D position of the ultrasound probe 122 that is located in an examination location of a target object, and forms position information. The sensing unit 718 may sense 3D movements, rotations, etc. of the ultrasound probe 122 to form position information. The sensed position information may include at least one of movement information, rotation information, and direction information of the ultrasound probe 122. The sensing unit 718 may be any device that may detect a 3D position of the ultrasound probe 122. For example, the sensing unit 718 may include at least one of an angular velocity sensor, a magnetic sensor, an accelerometer sensor, a gravity sensor, and a gyro sensor.

Based on the position information of the ultrasound probe 122, the processing unit 714 may generate an ultrasound beam direction marker that indicates a direction of an ultrasound beam that is transmitted from the ultrasound probe 122. The ultrasound beam direction marker may exactly coincide with a position or a direction of a probe, or different from the position or the direction of the probe due to steering or elevation. This will be described in detail below.

The transducer elements in the ultrasound probe 122 are sequentially arranged, and the ultrasound probe 122 transmits ultrasound signals by sequentially using the transducer elements. For example, when the transducer elements includes N transducer elements (a transducer element #1 through a transducer element #N, wherein N is an integer equal to 2 or more), the ultrasound probe 122 transmits an ultrasound signal via a transducer element #1, transmits an ultrasound signal via a transducer element #2, and then finally, transmits an ultrasound signal via a transducer element #N.

The processing unit 714 generates the ultrasound beam direction marker such that the ultrasound beam direction marker indicates directions in which the ultrasound signals are transmitted via the N transducer elements. The generated ultrasound beam direction marker may be similar to a rectangle or a sector which is formed in a three-dimensional space according to a shape of the ultrasound probe 122. When the ultrasound probe 122 is a convex probe type or a curved probe type similar to a convex probe type, a distance between the ultrasound signal transmitted via the transducer element #1 and the ultrasound signal transmitted via the transducer element #2 increases along a direction farther away from the ultrasound probe 122. Therefore, the ultrasound beam direction marker that is generated by such probe has a shape similar to a sector. Hereinafter, the ultrasound beam direction marker in such case is referred to as sector-shaped. When the ultrasound probe 122 is a linear probe, the ultrasound beam direction marker may be rectangular-shaped.

The processing unit 714 generates and displays a first indicator that indicates a reference point of the ultrasound beam direction marker. For example, the processing unit 714 may generate a first indicator that indicates a direction in which scan lines move as the ultrasound signals are sequentially transmitted via the transducer elements. The output unit 716 may display the ultrasound beam direction marker that is generated by the processing unit 714, and display the first indicator at a side of the ultrasound beam direction marker. The processing unit 714 may control the first indicator to be displayed at a particular position around the ultrasound beam direction marker so that a user may recognize that scanning is performed in a direction from a location of the transducer element #1 to the transducer element #N. The ultrasound beam direction marker and the first indicator may be overlapping each other or spaced apart from each other.

In one embodiment, the processing unit 714 may form an ultrasound image based on a plurality of scan lines formed by transmission of the ultrasound signals, and the output unit 716 may display the ultrasound image. In one embodiment, the processing unit 714 may display a second indicator at a side of the ultrasound image so that the second indicator indicates a location corresponding to the first indicator in the ultrasound beam direction marker. When the first indicator is displayed in the ultrasound beam direction marker at a location where scanning starts, the second indicator may be displayed in the ultrasound image at a location that corresponds to a scan line that is obtained via the transducer element #1 from among the plurality of scan lines, so that the second indicator in the ultrasound image is displayed at a location corresponding to the first indicator in the ultrasound beam direction marker. When the first indicator is displayed at a location in the ultrasound beam direction marker where scanning ends, in order to be displayed at a location corresponding to the first indicator, the second indicator may be displayed in the ultrasound image at a location that corresponds to a scan line that is obtained via the transducer element #N from among the plurality of scan lines. That is, the second indicator indicates a direction that corresponds to or is the same as a scanning direction indicated by the first indicator. The ultrasound image and the second indicator may overlap each other.

FIG. 7B is an exemplary diagram showing an illustrative embodiment of an ultrasound image 752 and an ultrasound beam direction marker 772.

The processing unit 714 generates an ultrasound beam direction marker 772 that indicates a direction in which ultrasound signals are transmitted via the transducer elements of the ultrasound probe 122. The output unit 716 displays the ultrasound beam direction marker 772. The ultrasound beam direction marker 772 may be sector-shaped as described above. A first line 774 of the sector corresponds to an ultrasound signal that is transmitted via the transducer element #1, i.e., a first transducer element from among the transducer elements, and forms a first scan line, and a second line 776 corresponds to an ultrasound signal that forms a last scan line that is transmitted via the transducer element #N, i.e., a last transducer element from among the transducer elements, and forms a last scan line. In other words, in the ultrasound beam direction marker 772, the ultrasound signals can be understood as starting to be transmitted through the first line 774 via the transducer element #1 and as lastly being transmitted through the second line 776 via the transducer element #N. Such ultrasound signal transmission process may be referred to as ‘scanning.’ In the ultrasound beam direction marker 772, scanning may be formed in a direction from the first line 774 to the second line 776.

The processing unit 714 may control the output unit 716 such that the output unit 716 displays the ultrasound beam direction marker 772 and the first indicator at a side of the ultrasound beam direction marker 772. The first indicator may indicate a reference point of the ultrasound beam direction marker 772. For example, the output unit 716 may display the first indicator 778 at a location corresponding to a scanning starting point. The reference point may be a location where scanning starts, i.e., a particular point on the first line 774. Alternatively, the first indicator may be displayed within a predetermined distance from the first line 774. According to a location where a first indicator 774 is displayed, a user of the ultrasound system 710 may recognize that a scanning direction is a direction from a first line 774 to a second line 776. A first indicator 778 may be any recognizable icon. For example, the first indicator 778 may be an icon including an alphabet.

The processing unit 714 may form the ultrasound image 752 by using echo signals that are reflected by the target object. The output unit 716 may display the ultrasound image 752. Furthermore, the processing unit 714 may control the output unit 716 such that the output unit 716 displays a second indicator 758 at a side of the ultrasound image 752, in order to indicate a location corresponding to a location of a first indicator shown at a side of the ultrasound beam direction marker 772. For example, when the first indicator 778 is displayed around the first line 774 to indicate a scanning start location in the ultrasound beam direction marker 772, the second indicator 758 may be displayed around a first line 754 formed at a scanning start point in the ultrasound image 752. The first indicator 778 and the second indicator 758 may be displayed within a predetermined distance from the first line 774 in the ultrasound beam direction marker 772 and the first line 754 in the ultrasound image 752.

Also, when the first indicator 778 is displayed at an end point of the first line 774 in the ultrasound beam direction marker 772, the second indicator 758 does not have to be displayed at an end point of the first line 754 in the ultrasound image 752, but may be displayed anywhere around, but within a particular distance from, the first line 754 in the ultrasound image. For example, when the first indicator 778 and the second indicator 758 have to indicate reference points where scanning starts, the first indicator 778 may be displayed at any location on or around the first line 774 of the ultrasound beam direction marker 772, and the second indicator 758 may be displayed at any location on or around the first line 754 of the ultrasound image 752.

According to an exemplary embodiment, by displaying the first indicator 778 at a side of the ultrasound beam direction marker 772 and displaying the second indicator 758 at a side of the ultrasound image 752 correspondingly to a location indicated by the first indicator 778, the user may intuitively recognize that by which direction of scanning a currently displayed ultrasound image is formed.

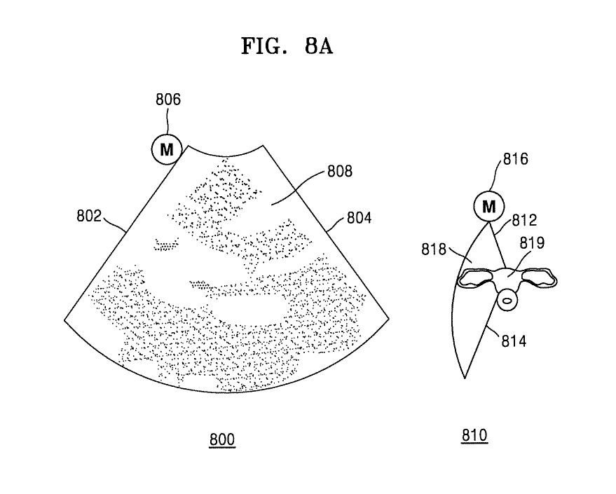

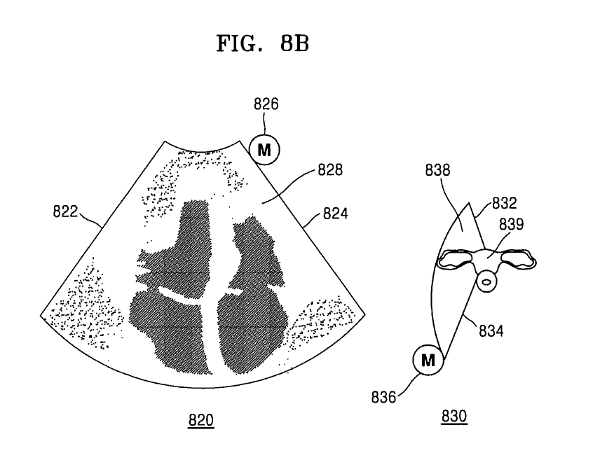

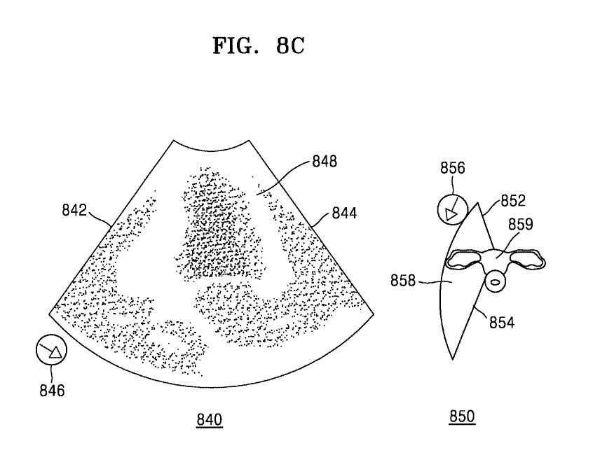

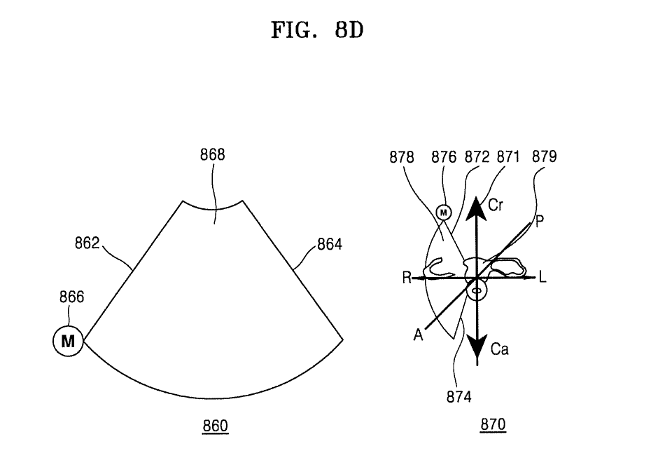

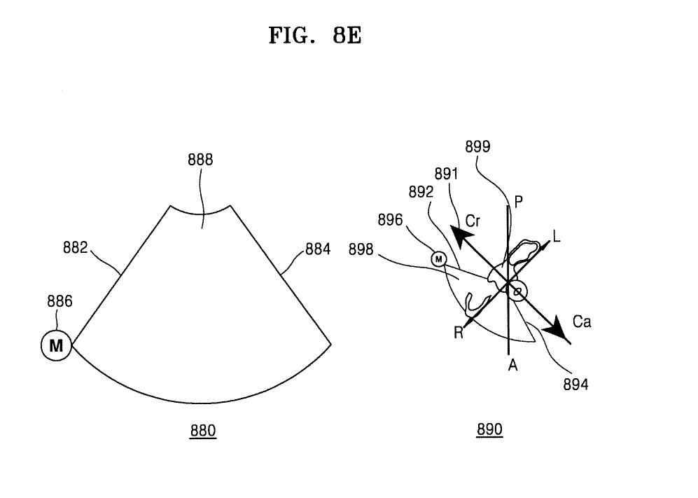

FIGS. 8A to 8E are exemplary diagrams showing various exemplary embodiments in which an ultrasound image and an ultrasound beam direction marker are displayed together.

As shown in FIGS. 8A to 8C, according to exemplary embodiments, the ultrasound system 710 may display an ultrasound image, an ultrasound beam direction marker, and a target organ marker. The target organ marker may overlap or spaced apart from the ultrasound beam direction marker. When the target organ marker and the ultrasound beam direction marker are displayed together, a user may intuitively understand that to which portion of the target organ marker is an ultrasound beam transmitted and that a cross-section of which target tissue is indicated by a displayed ultrasound image.

According to exemplary embodiments, the ultrasound system 710 may display a first indicator at a scanning start location or at a scanning end location. For example, as shown in FIG. 8A, the ultrasound system 710 displays an image 810 that includes a target organ marker 819, an ultrasound beam direction marker 818, and a first indicator 816. In the ultrasound beam direction marker 818, a first line 812 indicates a location where ultrasound signal transmission starts and a second line 814 indicates a location where ultrasound signal transmission ends. The first indicator 816 is displayed around the first line 812 that corresponds to the location where ultrasound signal transmission starts, for example, around an end of the first line 812, and thus, the first indicator 816 indicates that scanning is performed in a direction from a location of the first line 812 to a location of the second line 814.

Furthermore, in addition to the image 810 that indicates a direction in which ultrasound beam for scanning is transmitted, the ultrasound system 710 may display an image 800 that includes an ultrasound image 808 that is formed as a result of the scanning. The image 800 includes a second indicator 806 that indicates a predetermined reference point in the ultrasound image 808. A reference point or a reference direction that is indicated by the second indicator 806 in the ultrasound image 808 may correspond to a reference point or a reference direction indicated by the first indicator 816 in the ultrasound beam direction marker 818. Also, in the ultrasound image 808, a first line 802 indicates a scan line formed at a scanning start point and a second line 804 indicates a scan line formed at a scanning end point. In other words, in the image 800, scanning is performed in a direction from the first line 802 to the second line 804. In this case, the ultrasound system 710 may display the second indicator 806 on or around the first line 802 of the ultrasound image 808, and thus, indicate that a location of the first line 802 is a location where scanning starts and that scanning is performed in the direction from the first line 802 to the second line 804. A scanning direction indicated by the second indicator 806 is the same as or corresponds to a scanning direction indicated by the first indicator 816.

Referring to FIG. 8B, in one embodiment, the ultrasound system 710 may display an indicator at a scanning end location. For example, the ultrasound system 710 displays an image 830 that includes a target organ marker 839, an ultrasound beam direction marker 838, and a first indicator 836. Also, the ultrasound system 710 displays an image 820 which displays an ultrasound image 828 that is formed by scanning and a second indicator 826, as in the image 830. A first line 832 of the ultrasound beam direction marker 838 and a first line 822 of the ultrasound image 828 correspond to a location where ultrasound signal transmission starts, and a second line 834 of the ultrasound beam direction marker 838 and a second line 824 of the ultrasound image 828 correspond to a location where ultrasound signal transmission ends. In this case, the ultrasound system 710 may display the first indicator 836 on or within a predetermined distance from the second line 834 of the ultrasound beam direction marker 838 and display the second indicator 826 at a location of the second line 824 of the ultrasound image 828, and thus, indicate that locations respectively indicated by the first indicator 836 and the second indicator 826 are scanning end locations. According to the present embodiment, the ultrasound system 710 may indicate that scanning is performed from the first line 832 to the second line 834 in the ultrasound beam direction marker 838 and that scanning is performed from the first line 822 to the second line 824 in the ultrasound image 828.

Referring to FIG. 8C, in one embodiment, the ultrasound system 710 may display a scanning direction by using an arrow. For example, the ultrasound system 710 may display a first indicator 856 including an arrow in a ultrasound beam direction marker 858 in order to indicate that scanning is performed from a first line 852 to a second line 854 of the ultrasound beam direction marker 858. Also, the ultrasound system 710 may display a second indicator 846 at a side of an ultrasound image 848 to indicate a location corresponding to the first indicator 856 in the ultrasound image 848. For example, the ultrasound system 710 may display the second indicator 846 including an arrow in the ultrasound image 848, and thus, indicate that scanning is performed in a direction from a first line 842 to a second line 844 in the ultrasound image 848.

Referring to FIGS. 8D and 8E, in one embodiment, the ultrasound system 710 may display an ultrasound image, an ultrasound beam direction marker, a target organ marker, and also, a body axis marker.

Referring to FIG. 8D, the ultrasound system 710 displays a target organ marker 879 and a body axis marker 871 together. An anatomical location, direction, or orientation of the target organ marker 879 or an ultrasound beam direction marker 878 can be recognized by additionally displaying the body axis marker 871. For example, a direction or an orientation of a target organ marker 899 of FIG. 8E is relatively inclined compared to a direction or an orientation of the target organ marker 879 of FIG. 8D. In this case, by displaying the body axis marker 871 and a body axis marker 891 together with the target organ markers 879 and 899, the user may intuitively recognize anatomical directions or orientations of the target organ markers 879 and 899 and the ultrasound beam direction markers 878 and an ultrasound beam direction marker 898. As shown in FIGS. 8D and 8E, a body axis marker may be appropriately changed as a direction or an orientation of a displayed target organ marker changes.

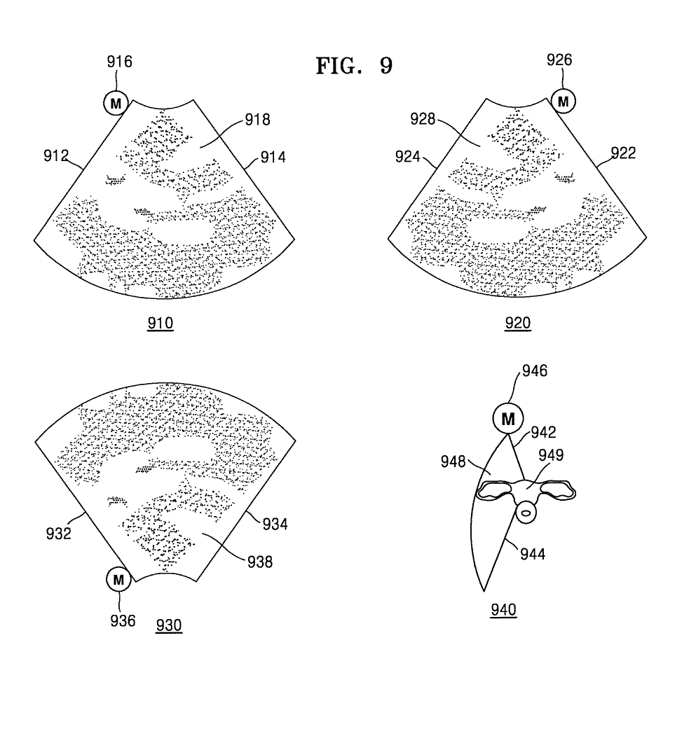

FIG. 9 is an exemplary diagram showing an illustrative embodiment of an ultrasound image and an ultrasound beam direction marker when the ultrasound image is symmetrically flipped vertically or horizontally.

As shown in an image 940, the ultrasound system 710 performs scanning on a target organ and obtains an ultrasound image. In this case, the image 940 displays a target organ marker 949, an ultrasound beam direction marker 948, and a first indicator 946 to indicate a scanning location and a scanning direction related to the target organ. For convenience, the image 940 is referred to as ‘marker image 940.’

The ultrasound system 710 forms an image of a certain view of a target organ by scanning and displays the formed image. For example, the ultrasound system 710 may form and display an ultrasound image 918 based on the scanning. An image 910 includes the ultrasound image 918 and a second indicator 916. For convenience, the image 910 is referred to as ‘reference image 910.’ The second indicator 916 shown in the reference image 910 indicates a location corresponding to the first indicator 946 shown in the marker image 940. For example, when a first line 942 of the ultrasound beam direction marker 948 in the marker image 940 is a location where ultrasound signal transmission starts and the first indicator 946 is displayed around the first line 942, a first line 912 of the ultrasound image 918 in the reference image 910 is a first scan line formed at a scanning start point, and the second indicator 916 may be displayed around the first line 912 of the ultrasound image 918. That is, a location indicated by the second indicator 916 in the reference image 910 may correspond to a location indicated by the first indicator 946 in the marker image 940. Accordingly, the first indicator 946 in the marker image 940 and the second indicator 916 in the reference image 910 may respectively indicate scanning directions in the marker image 940 and the reference image 910.

According to exemplary embodiments, the ultrasound system 710 may symmetrically flip the ultrasound image 918 in the reference image 910 vertically or horizontally if necessary. An image 920 shows the ultrasound image 918 that is symmetrically flipped horizontally. For convenience, the image 920 is referred to as ‘horizontally flipped image 920.’ In this case, in the horizontally flipped image 920, a second indicator 926 is displayed at a new location to indicate a flipped location. Based on the location of the second indicator 926 in the horizontally flipped image 920, the user may intuitively recognize that ultrasound scanning is performed in a direction from a first line 922 to a second line 924. According to exemplary embodiments, even when the ultrasound system 710 does not display the reference image 910 and only displays the marker image 940 and the horizontally flipped image 920, the user may check the first indicator 946 in the marker image 940 and the second indicator 926 in the horizontally flipped image 920, and thus intuitively recognize a scanning direction in the ultrasound marker image 940 and the horizontally flipped image 920. In this case, the first indicator 946 in the marker image 940 and the second indicator 926 in the horizontally flipped image 920 indicate corresponding locations.

In one embodiment, the ultrasound system 710 may symmetrically and vertically flip or reverse the reference image 910 and thus display a vertically flipped image 930. In this case, a second indicator 936 may be displayed at a location in the vertically flipped image 930 so that the location of the second indicator 936 corresponds to the location of the first indicator 946 of the marker image 940. The user may recognize that scanning is performed in a direction from a first line 932 to a second line 934 of an ultrasound image 938 by checking a location of the second indicator 936 in the vertically flipped image 930. When the reference image 910 is not displayed but only the marker image 940 and the vertically flipped image 930 are displayed, the user may check locations of the first indicator 946 in the marker image 940 and the second indicator 936 in the vertically flipped image 930, and thus intuitively recognize a scanning direction in the marker image 940 and the vertically flipped image 930. In this case, the first indicator 946 in the marker image 940 and the second indicator 936 in the vertically flipped image 930 indicate corresponding locations.

When a first indicator in an ultrasound beam direction marker and a second indicator in an ultrasound image are not displayed, with respect to a target organ, the user may recognize an ultrasound beam transmission direction but may have difficulty in directly recognizing a scanning direction. Also, the user may not be able to recognize a scanning direction in a displayed ultrasound image. According to the present inventive concept, the user may easily recognize a scanning direction in an ultrasound beam direction marker and an ultrasound image, and thus, the user may be less confused about which portion of a target organ is estimated and displayed in which manner.

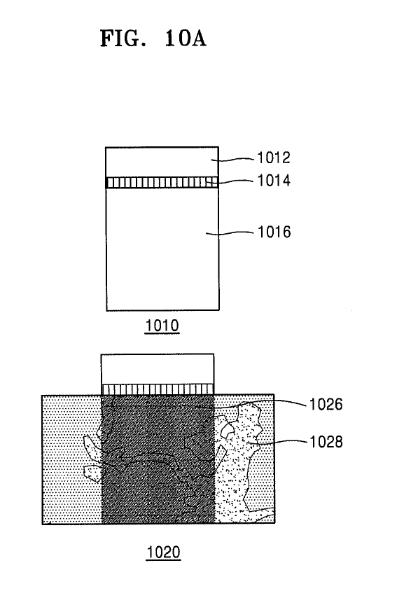

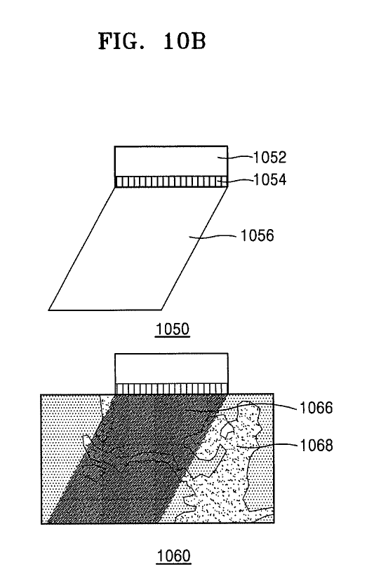

FIGS. 10A and 10B are exemplary diagrams showing an example of a transmission direction of an ultrasound signal being changed compared to a direction of a probe.

Referring to FIG. 10A, an image 1010 shows an ultrasound probe 1012, a plurality of transducer elements 1014 in the ultrasound probe 1012, and an ultrasound beam direction marker 1016 indicating a direction of ultrasound beams transmitted from the ultrasound probe 1012. Also, an image 1020 indicates an ultrasound beam direction marker 1026 and an ultrasound image 1028 that are displayed according to transmission of ultrasound beams as in the image 1010. According to the images 1010 and 1020, the ultrasound beams pass through the ultrasound probe 1012 to the transducer elements 1014, transmitted in an original transmission direction, penetrate through a certain cross-section of a target organ, and thus, the ultrasound system 710 forms the ultrasound image 1028.

On the other hand, referring to FIG. 10B, ultrasound beams are not transmitted in an original transmission direction from an ultrasound probe 1052 but a transmission direction has been steered or elevated. Directions and locations of the ultrasound probe 1052 and a plurality of transducer elements 1054 in an image 1050 are the same as those in the image 1010. However, after the ultrasound beams pass through the ultrasound probe 1052 to the transducer elements 1054, the original transmission direction is not maintained but is slightly steered or elevated leftward. Accordingly, referring to an image 1060, the ultrasound beams are transmitted to a different plane of the same target organ compared to the image 1020. Therefore, an ultrasound image 1068 formed in FIG. 10B is somewhat different from the ultrasound image 1028 of FIG. 10A.

As shown in FIGS. 10A and 10B, even with a probe and transducer elements of the same locations and directions, ultrasound beams may be transmitted in different directions by steering or elevating. Therefore, in order to know an actual portion of a target organ through which the ultrasound beams passed, it is necessary to identify a direction the ultrasound beams that actually penetrates through the target organ, instead of identifying locations and directions of ultrasound probes and transducer elements. The ultrasound beam direction markers described with reference to FIGS. 1 to 9C do not indicate a location and a direction of an ultrasound probe but locations and directions of ultrasound beams that are actually transmitted toward a target organ.

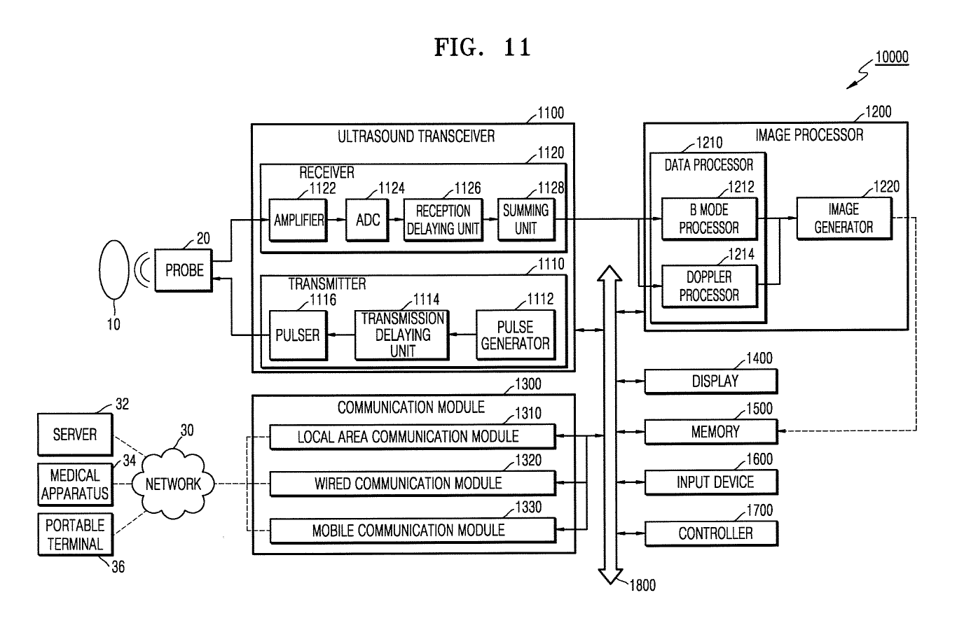

FIG. 11 is a block diagram showing a configuration of an ultrasound diagnosis apparatus 10000 according to an embodiment. Referring to FIG. 11, the ultrasound diagnosis apparatus 10000 may include a probe 20, an ultrasound transceiver 1100, an image processor 1200, a communication module 1300, a display 1400, a memory 1500, an input device 1600, and a controller 1700, which may be connected to one another via buses 1800.

The ultrasound system 100 according to an exemplary embodiment of FIG. 1 may be included in the ultrasound diagnosis apparatus 10000. The ultrasound system 100 may perform some or all functions of the ultrasound diagnosis apparatus 10000. For example, the ultrasound data acquisition unit 120 of FIG. 1 includes some components or functions of the ultrasound transceiver 1100 and the probe 20 of FIG. 11. Also, the ultrasound image forming unit 130, the processing unit 160, and the control unit 180 of FIG. 1 include some components or functions of the image processor 1200 and the controller 1700 of FIG. 11. Also, the input unit 110, the storage unit 140, and the output unit 170 of FIG. 1 respectively correspond to the input device 1600, the memory 1500, and the display 1400 of FIG. 11.

The ultrasound system 710 according to an exemplary embodiment of FIG. 7A may be included in the ultrasound diagnosis apparatus 10000. The ultrasound system 710 may perform some or all functions of the ultrasound diagnosis apparatus 10000. For example, the ultrasound data acquisition unit 712 and the output unit 716 of FIG. 7A respectively correspond to the ultrasound transceiver 1100 and the display 1400 of FIG. 11. Also, the processing unit 714 of FIG. 7A may include some components of the image processor 1200 and the controller 1700 of FIG. 11.

The ultrasound diagnosis apparatus 10000 may be a cart type apparatus or a portable type apparatus. Examples of portable ultrasound diagnosis apparatuses may include, but are not limited to, a picture archiving and communication system (PACS) viewer, a smartphone, a laptop computer, a personal digital assistant (PDA), and a tablet PC.

The probe 20 transmits ultrasound waves to a target object 10 in response to a driving signal applied by the ultrasound transceiver 1100 and receives echo signals reflected by the target object 10. The probe 20 includes a plurality of transducers, and the plurality of transducers oscillate in response to electric signals and generate acoustic energy, that is, ultrasound waves. Furthermore, the probe 20 may be connected to the main body of the ultrasound diagnosis apparatus 10000 by wire or wirelessly, and according to embodiments, the ultrasound diagnosis apparatus 10000 may include a plurality of probes 20.

A transmitter 1110 supplies a driving signal to the probe 20. The transmitter 110 includes a pulse generator 1112, a transmission delaying unit 1114, and a pulser 1116. The pulse generator 1112 generates pulses for forming transmission ultrasound waves based on a predetermined pulse repetition frequency (PRF), and the transmission delaying unit 1114 delays the pulses by delay times necessary for determining transmission directionality. The pulses which have been delayed correspond to a plurality of piezoelectric vibrators included in the probe 20, respectively. The pulser 1116 applies a driving signal (or a driving pulse) to the probe 20 based on timing corresponding to each of the pulses which have been delayed.

A receiver 1120 generates ultrasound data by processing echo signals received from the probe 20. The receiver 120 may include an amplifier 1122, an analog-to-digital converter (ADC) 1124, a reception delaying unit 1126, and a summing unit 1128. The amplifier 1122 amplifies echo signals in each channel, and the ADC 1124 performs analog-to-digital conversion with respect to the amplified echo signals. The reception delaying unit 1126 delays digital echo signals output by the ADC 1124 by delay times necessary for determining reception directionality, and the summing unit 1128 generates ultrasound data by summing the echo signals processed by the reception delaying unit 1126. In some embodiments, the receiver 1120 may not include the amplifier 1122. In other words, if the sensitivity of the probe 20 or the capability of the ADC 1124 to process bits is enhanced, the amplifier 1122 may be omitted.

The image processor 1200 generates an ultrasound image by scan-converting ultrasound data generated by the ultrasound transceiver 1100. The ultrasound image may be not only a grayscale ultrasound image obtained by scanning a target object in an amplitude (A) mode, a brightness (B) mode, and a motion (M) mode, but also a Doppler image showing a movement of a target object via a Doppler effect. The Doppler image may be a blood flow Doppler image showing flow of blood (also referred to as a color Doppler image), a tissue Doppler image showing a movement of tissue, or a spectral Doppler image showing a moving speed of a target object as a waveform.

A B mode processor 1212 extracts B mode components from ultrasound data and processes the B mode components. An image generator 1220 may generate an ultrasound image indicating signal intensities as brightness based on the extracted B mode components 1212.

Similarly, a Doppler processor 1214 may extract Doppler components from ultrasound data, and the image generator 1220 may generate a Doppler image indicating a movement of a target object as colors or waveforms based on the extracted Doppler components.

According to an embodiment, the image generator 1220 may generate a three-dimensional (3D) ultrasound image via volume-rendering with respect to volume data and may also generate an elasticity image by imaging deformation of the target object 10 due to pressure. Furthermore, the image generator 1220 may display various pieces of additional information in an ultrasound image by using text and graphics. In addition, the generated ultrasound image may be stored in the memory 1500.

A display 1400 displays the generated ultrasound image. The display 1400 may display not only an ultrasound image, but also various pieces of information processed by the ultrasound diagnosis apparatus 10000 on a screen image via a graphical user interface (GUI). In addition, the ultrasound diagnosis apparatus 10000 may include two or more displays 1400 according to embodiments.

The communication module 1300 is connected to a network 30 by wire or wirelessly to communicate with an external device or a server. The communication module 1300 may exchange data with a hospital server or another medical apparatus in a hospital, which is connected thereto via a PACS. Furthermore, the communication module 1300 may perform data communication according to the digital imaging and communications in medicine (DICOM) standard.

The communication module 1300 may transmit or receive data related to diagnosis of a target object, e.g., an ultrasound image, ultrasound data, and Doppler data of the target object, via the network 30 and may also transmit or receive medical images captured by another medical apparatus, e.g., a computed tomography (CT) apparatus, a magnetic resonance imaging (MRI) apparatus, or an X-ray apparatus. Furthermore, the communication module 1300 may receive information about a diagnosis history or medical treatment schedule of a patient from a server and utilizes the received information to diagnose the patient. Furthermore, the communication module 1300 may perform data communication not only with a server or a medical apparatus in a hospital, but also with a portable terminal of a medical doctor or patient.

The communication module 1300 is connected to the network 30 by wire or wirelessly to exchange data with a server 32, a medical apparatus 34, or a portable terminal 36. The communication module 1300 may include one or more components for communication with external devices. For example, the communication module 1300 may include a local area communication module 1310, a wired communication module 1320, and a mobile communication module 1330.

The local area communication module 1310 refers to a module for local area communication within a predetermined distance. Examples of local area communication techniques according to an embodiment may include, but are not limited to, wireless LAN, Wi-Fi, Bluetooth, ZigBee, Wi-Fi Direct (WFD), ultra wideband (UWB), infrared data association (IrDA), Bluetooth low energy (BLE), and near field communication (NFC).

The wired communication module 1320 refers to a module for communication using electric signals or optical signals. Examples of wired communication techniques according to an embodiment may include communication via a twisted pair cable, a coaxial cable, an optical fiber cable, and an Ethernet cable.

The mobile communication module 1330 transmits or receives wireless signals to or from at least one selected from a base station, an external terminal, and a server on a mobile communication network. The wireless signals may be voice call signals, video call signals, or various types of data for transmission and reception of text/multimedia messages.

The memory 1500 stores various data processed by the ultrasound diagnosis apparatus 10000. For example, the memory 1500 may store medical data related to diagnosis of a target object, such as ultrasound data and an ultrasound image that are input or output, and may also store algorithms or programs which are to be executed in the ultrasound diagnosis apparatus 10000.

The memory 1500 may be any of various storage media, e.g., a flash memory, a hard disk drive, EEPROM, etc. Furthermore, the ultrasound diagnosis apparatus 10000 may utilize web storage or a cloud server that performs the storage function of the memory 400 online.

The input device 1600 refers to a means via which a user inputs data for controlling the ultrasound diagnosis apparatus 10000. The input device 1600 may include hardware components, such as a keypad, a mouse, a touch pad, a touch screen, and a jog switch. However, embodiments are not limited thereto, and the input device 1600 may further include any of various other input units including an electrocardiogram (ECG) measuring module, a respiration measuring module, a voice recognition sensor, a gesture recognition sensor, a fingerprint recognition sensor, an iris recognition sensor, a depth sensor, a distance sensor, etc.

The controller 1700 may control all operations of the ultrasound diagnosis apparatus 10000. In other words, the controller 1700 may control operations among the probe 20, the ultrasound transceiver 1100, the image processor 1200, the communication module 1300, the display 1400, the memory 1500, and the input device 1600 shown in FIG. 11.

All or some of the probe 20, the ultrasound transceiver 1100, the image processor 1200, the communication module 1300, the display 1400, the memory 1500, the input device 1600, and the controller 1700 may be implemented as software modules. Also, at least one of the ultrasound transmission/reception unit 1100, the image processor 1200, and the communication module 1300 may be included in the control unit 1600; however, the inventive concept is not limited thereto.

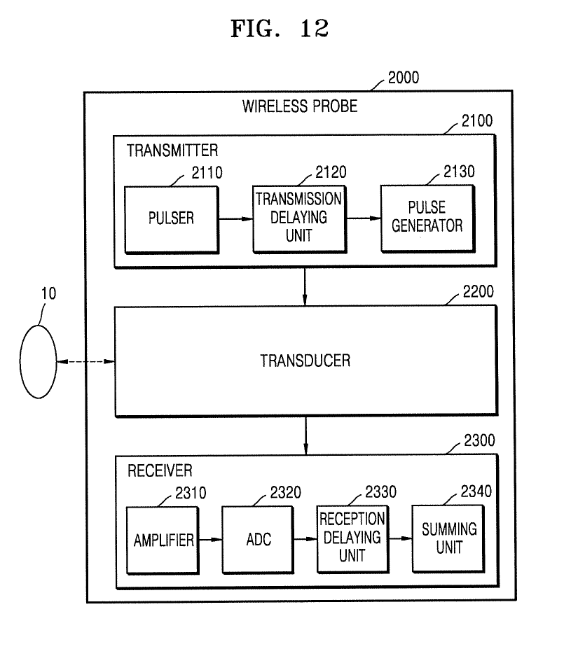

FIG. 12 is a block diagram showing a configuration of a wireless probe 2000 according to an embodiment. As described above with reference to FIG. 11, the wireless probe 2000 may include a plurality of transducers, and, according to embodiments, may include some or all of the components of the ultrasound transceiver 1100 shown in FIG. 11.

The wireless probe 2000 according to the embodiment shown in FIG. 12 includes a transmitter 2100, a transducer 2200, and a receiver 2300. Since descriptions thereof are given above with reference to FIG. 11, detailed descriptions thereof will be omitted here. In addition, according to embodiments, the wireless probe 2000 may selectively include a reception delaying unit 2330 and a summing unit 2340.

The wireless probe 2000 may transmit ultrasound signals to the target object 10, receive echo signals from the target object 10, generate ultrasound data, and wirelessly transmit the ultrasound data to the ultrasound diagnosis apparatus 10000 shown in FIG. 11.

While the present invention is described by some preferred embodiments, it will be appreciated by those skilled in the art that many modifications and changes can be made without departing from the spirit and scope of the appended claims. It will be understood by those of ordinary skill in the art that various changes in form and details may be made therein without departing from the spirit and scope of the inventive concept as defined by the following claims.

What is claimed is:

1. An ultrasound system comprising:
a probe scanning a target object by transmitting ultrasound signals and receiving ultrasound echo signals;
a processor forming an ultrasound image based on the ultrasound echo signals and an ultrasound beam direction marker indicating a transmission direction of the ultrasound signals; and
a display displaying the ultrasound image and the ultrasound beam direction marker,
wherein the processor controls the display to display a first indicator indicating a reference point of the ultrasound beam direction marker at a side of the ultrasound beam direction marker, and a second indicator indicating a location corresponding to the first indicator at a side of the ultrasound image.

2. The ultrasound system of claim 1, wherein the display further displays a target object marker indicating the target object, and
a display direction of the ultrasound beam direction marker with respect to the target object marker corresponds to a transmission direction of the ultrasound signals with respect to the target object.

3. The ultrasound system of claim 1, wherein the first indicator is displayed in the ultrasound beam direction marker at a location where the scanning starts or within a predetermined distance from the location where the scanning starts.

4. The ultrasound system of claim 3, wherein the processor controls the display to display the second indicator at a location corresponding to a scan line where the scanning starts, from among a plurality of scan lines comprised in the ultrasound image.

5. The ultrasound system of claim 1, wherein the first indicator is displayed in the ultrasound beam direction marker at a location where the scanning ends or within a predetermined distance from the location where the scanning ends.

6. The ultrasound system of claim 5, wherein the processor controls the display to display the second indicator at a location corresponding to a scan line where the scanning ends, from among a plurality of scan lines comprised in the ultrasound image.

7. The ultrasound system of claim 1, wherein the first indicator indicates a direction of the scanning in the ultrasound beam direction marker by using a first arrow.

8. The ultrasound system of claim 7, wherein the second indicator indicates a direction of the scanning in the ultrasound image by using a second arrow.

9. The ultrasound system of claim 1, wherein the display displays a symmetrically flipped image of the ultrasound image vertically or horizontally, and the second indicator indicates a location changed according to a direction in which the ultrasound image is symmetrically flipped.

10. A method of displaying an ultrasound image, the method comprising:
scanning a target object by transmitting ultrasound signals and receiving ultrasound echo signals;
forming an ultrasound image based on the ultrasound echo signals and an ultrasound beam direction marker indicating a transmission direction of the ultrasound signals; and
displaying the ultrasound image and the ultrasound beam direction marker, displaying a first indicator indicating a reference point of the ultrasound beam direction marker at a side of the ultrasound beam direction marker, and displaying a second indicator indicating a location corresponding to the first indicator at a side of the ultrasound image.

11. The method of claim 10, further comprising displaying a target object marker indicating the target object, and
wherein a display direction of the ultrasound beam direction marker with respect to the target object marker corresponds to a transmission direction of the ultrasound signals with respect to the target object.

12. The method of claim 10, wherein the first indicator is displayed in the ultrasound beam direction marker at a location where the scanning starts or within a predetermined distance from the location where the scanning starts.

13. The method of claim 12, further comprising displaying the second indicator at a location corresponding to a scan line where the scanning starts, from among a plurality of scan lines comprised in the ultrasound image.

14. The method of claim 10, wherein the first indicator is displayed in the ultrasound beam direction marker at a location where the scanning ends or within a predetermined distance from the location where the scanning ends.

15. The method of claim 14, further comprising displaying the second indicator at a location corresponding to a scan line where the scanning ends, from among a plurality of scan lines comprised in the ultrasound image.

16. The method of claim 10, wherein the first indicator indicates a direction of the scanning in the ultrasound beam direction marker by using a first arrow.

17. The method of claim 16, wherein the second indicator indicates a direction of the scanning in the ultrasound image by using a second arrow.

18. The method of claim 10, wherein the ultrasound image is symmetrically flipped vertically or horizontally and displayed, and the second indicator indicates a location changed according to a direction in which the ultrasound image is symmetrically flipped.

19. A non-transitory computer-readable recording medium having recorded thereon a program, which, when executed by a computer, performs the method of claim 10.

20. An ultrasound system comprising:
a probe scanning a target object by transmitting ultrasound signals and receiving ultrasound echo signals;
a processor forming an ultrasound image based on the ultrasound echo signals, an ultrasound beam direction marker indicating a transmission direction of the ultrasound signals, and a first indicator indicating a scanning direction of the scanning; and
a display displaying the ultrasound image, the ultrasound beam direction marker, and the first indicator.
